(19) United States
(12) Patent Application Publication
(10) Pub. No.: US 2013/0023769 A1
(43) Pub. Date: Jan. 24, 2013

(54) Tissue Penetration Device Coupled with Ultrasound Scanner

(71) Applicants: Ming Lin Julius Tsai, Singapore (SG); Chuen Neng Lee, Singapore (SG); Jui Lim, Singapore (SG)

(72) Inventors: Ming Lin Julius Tsai, Singapore (SG); Chuen Neng Lee, Singapore (SG); Jui Lim, Singapore (SG)

(21) Appl. No.: 13/574,853

(22) PCT Filed: Jan. 25, 2011
(86) PCT No.: PCT/SG11/00031

§ 371 Date: Oct. 15, 2012

Related U.S. Application Data
(60) Provisional application No. 61/297,839, filed on Jan. 25, 2010.

(51) Int. Cl. A61B 8/00

(52) U.S. Cl. 600/461

(57) ABSTRACT

Embodiments provide a device for penetrating a tissue of a subject. The device comprises a tissue penetrating portion having a tissue penetrating end. The device further comprises an ultrasonic scanner coupled to the tissue penetrating portion. The ultrasonic scanner comprises an ultrasound transduction circuit being configured to generate and transmit ultrasound waves to the subject and to receive returned sound waves echoed from the subject. The ultrasonic scanner further comprises a pulse generating circuit being configured to generate an electrical pulse so that the ultrasound transduction circuit is triggered to generate and transmit ultrasound waves.

The present application claims the benefit of the U.S. provisional patent application 61/297,839 (filed on 25 Jan. 2010), the entire contents of which are incorporated herein by reference for all purposes.

### TECHNICAL FIELD

Embodiments relate generally to a device for penetrating a tissue of a subject.

### BACKGROUND

In surgical operation, there is a need to inject anesthetic into the body. During procedures such as epidural anesthesia and general anesthesia, the difficulty in the procedure is the requirement of finding the right spot for intervention. In general anesthesia, the anesthetic is injected into the body through the artery. Before the intervention, the anesthetist normally uses an ultrasound imager to identify the artery location and make a mark on the patient. After that, the ultrasound imager is removed and the anesthetist will have to rely on the feeling and experience such as the back flow of the blood into the syringe to clearly know that the needle has reached the right location, meaning into the artery. On the other hand, procedures such as epidural anesthesia do not have this privilege as blood back flow and ultrasound imaging. This is because that there is essentially no blood in spinal cord and the ultrasound cannot penetrate bones. Therefore the procedure is even more challenging for the practitioners.

In another aspect, during the procedure of amniocentesis or chorionic villus sampling, samples are taken from the amniotic fluid or placenta via an intervention device such as a needle or catheter. There are however risks that the intervention device may accidentally penetrate the fetus. Similarly, during the procedure of organ tissue sampling such as liver tissue sampling, there are risks that the intervention device for collecting the tissue samples may accidentally penetrate artery.

### SUMMARY

Various embodiments provide a device for penetrating a tissue of a subject which solves at least partially the above mentioned problems.

In one embodiment, a device for penetrating a tissue of a subject is provided. The device may include a tissue penetrating portion having a tissue penetrating end. The device may further include an ultrasonic scanner coupled to the tissue penetrating portion. The ultrasonic scanner may include an ultrasound transduction circuit being configured to generate and transmit ultrasound waves to the subject and to receive returned sound waves echoed from the subject. The ultrasonic scanner may further include a pulse generating circuit being configured to generate an electrical pulse so that the ultrasound transduction circuit is triggered to generate and transmit ultrasound waves.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 (a) illustrates a device for penetrating a tissue of a subject in one exemplary embodiment;

FIG. 1 (b) illustrates the cross section view of the subject facing end of the device as shown in FIG. 1 (a);

FIG. 1 (c) illustrates the working mechanism of an ultrasound transduction circuit according to one exemplary embodiment;

FIG. 2 (a) illustrates a photo of a device for penetrating a tissue of a subject in one exemplary embodiment;

FIG. 2 (b) illustrates a photo of the device as shown in FIG. 2 (a) which is dissembled;

FIG. 3 (a) illustrates that an application-specific integrated circuit (ASIC) and a ultrasound transducer are electrically interconnected with a circuit board via wire-bond;

FIG. 3 (b) illustrates that the bond wire, the ASIC and part of the miniaturized ultrasound transducer are molded for protection;

FIG. 3 (c) illustrates the circuit board is bended at the portion of the ultrasound transducer and a back support plate is glued at the back of the circuit board to increase the rigidity of the circuit board;

FIG. 4 (a) illustrates that the electrical interconnection between the circuit board and the ASIC is achieved by flip-chip, and the electrical interconnection between the circuit board and the ultrasound transducer is also achieved by flip-chip; and

FIG. 4 (b) illustrates that mixed types of electrical interconnection between the circuit board and the ASIC and between the circuit board and the ASIC are applied.

### DESCRIPTION

The following detailed description refers to the accompanying drawings that show, by way of illustration, specific details and embodiments in which the invention may be practiced. These embodiments are described in sufficient detail to enable those skilled in the art to practice the invention. In this regard, directional terminology, such as “top”, “bottom”, “front”, “back”, “leading”, “trailing”, etc, is used with reference to the orientation of the Figure(s) being described. Because components of embodiments can be positioned in a number of different orientations, the directional terminology is used for purposes of illustration and is in no way limiting. Other embodiments may be utilized and structural, logical, and electrical changes may be made without departing from the scope of the invention. The various embodiments are not necessarily mutually exclusive, as some embodiments can be combined with one or more other embodiments to form new embodiments. The following detailed description therefore, is not to be taken in a limiting sense, and the scope of the present invention is defined by the appended claims.

The word “exemplary” is used herein to mean “serving as an example, instance, or illustration”. Any embodiment or design described herein as “exemplary” is not necessarily to be construed as preferred or advantageous over other embodiments or designs.

Embodiments provide a device for penetrating a tissue of a subject. For example, the subject may be a person or an animal. The device may include a tissue penetrating portion having a tissue penetrating end. The device may be configured to penetrate the tissue via the tissue penetrating end. The device may further include an ultrasonic scanner coupled to the tissue penetrating portion. The ultrasonic scanner may include an ultrasound transduction circuit which is configured to generate and transmit ultrasound waves to the subject and to receive returned sound waves echoed from the subject. The ultrasonic scanner may further include a pulse generating circuit which is configured to generate an electrical pulse so that the ultrasound transduction circuit is triggered to generate and transmit ultrasound waves.

In other words, in one embodiment, the device may be used to penetrate a tissue of a subject, e.g. a patient, via the tissue penetration portion, e.g. a needle or catheter. For example, the device may be used in the procedures of anesthesia such as epidural anesthesia, and anesthetic may be injected into the body through the artery or spinal cord using the device. As mentioned earlier, when injecting the anesthetic into an artery, the anesthetist may rely partially on the help of an ultrasound imager but is still required to rely on feeling and experience to clearly know that the needle has reached the right location. When injecting anesthetic into spinal cord, the procedure becomes more challenging because the ultrasound can not penetrate bones. The device as described herein includes an ultrasonic scanner which is configured to generate and transmit ultrasound waves and to receive returned sound waves echoed from the subject. The device may be configured to be coupled to a processor optionally with a monitor, for example. The returned sound waves may vibrate transducer(s) (which may be included in the ultrasound transduction circuit), and the transducer(s) may turn the vibrations into electrical pulses that may travel to the processor where the electrical pulses may be processed and transformed into a digital image and displayed on the monitor. Accordingly, by coupling such an ultrasonic scanner to the tissue penetrating portion, the anesthetist may determine from the image whether the tissue penetrating portion, e.g. a needle or a catheter, has reached the right location for the injection of anesthetic. It is appreciated that the application of the device in the procedure of anesthesia described herein is only for illustration purpose but should not be limited thereto.

For another example, the device as described herein may be applied during the procedure of amniocentesis or chorionic villus sampling. As mentioned earlier, in these procedures, samples are taken from the amniotic fluid or placenta via an intervention device such as a needle or catheter, and there are risks that the intervention device may accidentally penetrate the fetus. The device as described herein may help the practitioners to accurately locate the precise location to collect the sample from amniotic fluid or placenta without damaging vulnerable fetus. Similarly, the device as described herein may be applied in the procedures of organ tissue sampling, e.g. liver tissue sampling.

In other words, the device for penetrating a tissue of a subject as described herein may facilitate both the procedure of drug delivery, e.g. anesthetic delivery, and the procedure of collecting tissue samples.

In one embodiment, the tissue penetrating portion is of an elongated shape. For example, the tissue penetrating portion may be a needle or catheter which has an elongated shape.

In one embodiment, the tissue penetrating end is of a bevel shape. The bevel shape may facilitate the penetration of the device in the tissue of the subject.

In one exemplary embodiment, tissue penetrating portion may be a needle or a catheter. In another exemplary embodiment, the tissue penetrating portion may be a tube. In a further exemplary embodiment, the tissue penetrating portion may be a rod.

In one embodiment, ultrasonic scanner is of an elongated shape. For example, the elongated ultrasonic scanner may be coupled along to the elongated tissue penetrating portion. In the embodiment wherein the tissue penetrating portion has a hollow passway, the ultrasonic scanner may be inserted into the tissue penetrating portion and fitted alone the inside wall of the tissue penetrating portion.

In one embodiment, the ultrasonic scanner further includes an amplifying circuit being configured to amplify the returned sound waves. In a further embodiment, the ultrasonic scanner further includes a circuit board. For example, the circuit board may be a flexible circuit board which is bendable. For example, the circuit board may be of an elongated shape. In a still further embodiment, the ultrasound transduction circuit, the pulse generating circuit, and the amplifying circuit are coupled with the circuit board such that the ultrasound transduction circuit, the pulse generating circuit, and the amplifying circuit are electrically connected with each other via the circuit board.

In one embodiment, the pulse generating circuit and the amplifying circuit may be assembled together in an application-specific integrated circuit (ASIC), and the ASIC may be coupled to the circuit board such that the ASIC and the ultrasound transduction circuit may be electrically connected. The ASIC generally refers to an integrated circuit (IC) customized for a particular use.

In one embodiment, the circuit board may be coupled along the tissue penetrating portion.

In one embodiment, the ultrasound transduction circuit may be coupled to a subject facing end of the ultrasonic scanner. Accordingly, during the intervention procedure, field of view right at the tip (subject facing end) of device, e.g. a needle or catheter, may be provided. In a further embodiment, the subject facing end of the ultrasonic scanner is coupled to the tissue penetrating end of the tissue penetrating portion. Accordingly, when the device is penetrated into the tissue of the subject, the ultrasound transduction circuit may be configured to transmit ultrasound waves to and to receive returned sound waves from the portion of the tissue in front of the tissue penetrating end such that an image of the portion of the tissue in front of the tissue penetrating portion may be obtained. Based on the image, it may be determined whether the device has reached the right location. This may for example facilitate the practitioners a better control in precisely locating the spot to deliver medicine or anesthetic agents or to collect organ tissue samples.

In one embodiment, the ultrasound transduction circuit is coupled to a subject facing end of the circuit board. In a further embodiment, the subject facing end of the circuit board is coupled to the tissue penetrating end of the tissue penetrating portion.

In one embodiment, any of the ultrasound transduction circuit, the pulse generating circuit, and the amplifying circuit may be coupled to the circuit board via wire-bond or with flip-chip. For example, the ASIC may be coupled to the circuit board via wire-bond or with flip-chip.

In one embodiment, the ultrasound transduction circuit includes an array of ultrasound transducers. In a further embodiment, the ultrasound transduction circuit includes a linear array of one or more ultrasound transducers.

In one embodiment, the tissue penetrating portion is hollow, and the ultrasonic scanner may be fixed inside and along the tissue penetrating portion. In a further embodiment, the ultrasonic scanner may be fixed inside and along the tissue penetrating portion such that there is at least one passway through the tissue penetrating portion. Hollow space or passway between the tissue penetrating portion such as a needle and ultrasonic scanner may provide passage for drug delivery or for collecting tissue samples.

In one exemplary embodiment, the ultrasonic scanner is fixed inside and along the tissue penetrating portion using glue.

In the embodiment wherein the tissue penetrating portion is a rod, the ultrasonic scanner may be coupled along the rod. In a further embodiment, the ultrasound transduction circuit may be coupled to a subject facing end of the ultrasonic scanner and the subject facing end of the ultrasonic scanner may be coupled to the tissue penetrating end. In a still further embodiment, the rod and the ultrasonic scanner are configured to be covered with a sheath catheter upon penetrating the tissue of the subject. The space between the sheath catheter and the tissue penetrating portion may provide passage for drug delivery or for collection of tissue samples, for example.

In one embodiment, the device is configured to be coupled to an injector. For example, the injector may contain the drug to be delivered, and when it is determined that the device has reached the right location, the drug may be injected into the right location of the subject. For another example, sample tissues may be collected into the injector.

In one embodiment, the ultrasonic scanner is configured to receive a signal to initiate the pulse generating circuit to generate the electrical pulse.

In one embodiment, the ultrasonic scanner is configured to output the returned sound waves. For example, the returned sound waves may be turned into electrical pulses and may be further transmitted to a processor for further processing such that an image corresponding to the returned sound waves may be obtained. The practitioner may determine from the image whether the right location has been reached.

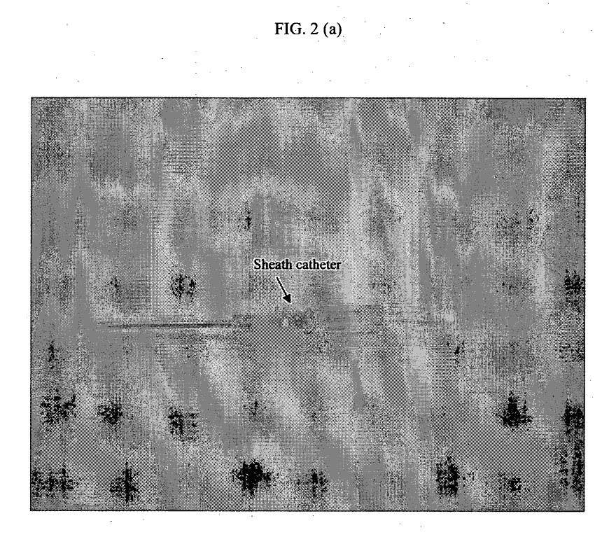

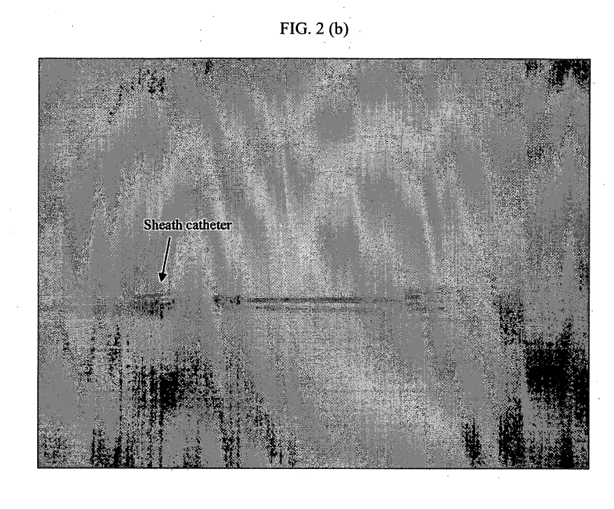

In other words, in one embodiment, the tissue penetrating portion may be a needle or catheter with bevel shape at the tip. Inside the needle or catheter contains the ultrasonic scanner. The ultrasonic scanner may include an ultrasound transduction circuit and a pulse generating circuit. The ultrasound transduction circuit may contain an ultrasound transducer array which may be attached to an ASIC for driving and signal amplification. The pulse generating circuit may be assembled in the ASIC. The ultrasonic scanner may further include an amplifying circuit which is configured to amplify the returned sound waves. The amplifying circuit may also be assembled in the ASIC. The ASIC and the ultrasound transduction circuit may be coupled to a circuit board. The ultrasound transduction circuit, the ASIC and the circuit board may only occupy a fraction of the cross-section of the tissue penetrating portion, e.g. a needle or catheter, so that there is passage to deliver medicine or anesthetic agents or to collect organ tissue samples. The needle or catheter itself may be hollow as a tube or solid as a rod. In the embodiment where the needle or catheter is a solid rod, a sheath catheter may be attached outside the needle or catheter for delivering the medicine or anesthetic agent or for collection of organ tissue samples, which is further illustrated with reference to FIGS. 2 (a) to (b). The sheath catheter may be left inside the intervention spot for drug delivery or vital sign monitoring purpose, for example.

The electrical signal and power may be fed through the circuit board inside the needle or catheter to external instrument for scan control and image processing. The ASIC may be only responsible for generating high voltage pulse to the ultrasound transduction circuit and amplifying received echo (returned waves) from the ultrasound transduction circuit. Therefore the cost of the ASIC may be reduced. This may make the device as described herein more competitive in disposable usage.

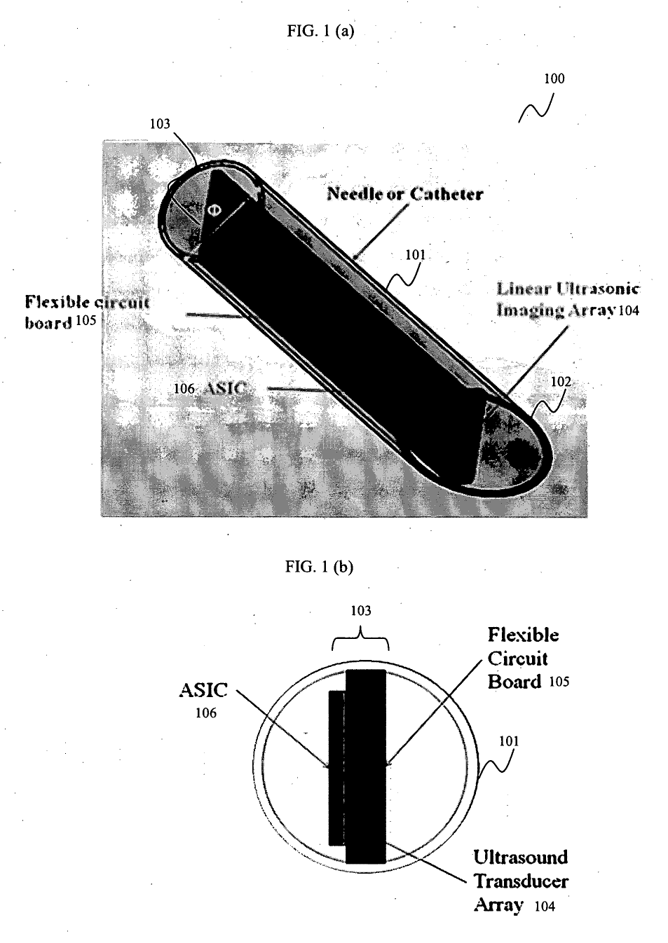

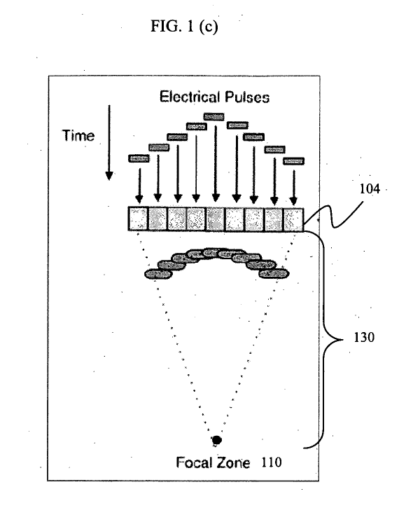

FIG. 1 (a) illustrates a device 100 for penetrating a tissue of a subject (not shown) in one exemplary embodiment.

The device 100 includes a tissue penetrating portion 101 having a tissue penetrating end 102. The device 100 may be configured to penetrate the tissue via the tissue penetrating end 102. The device 100 may further include an ultrasonic scanner 103 coupled to the tissue penetrating portion 101. The ultrasonic scanner 103 may include an ultrasound transduction circuit 104 which is configured to generate and transmit ultrasound waves to the subject and to receive returned sound waves echoed from the subject. The ultrasonic scanner 103 may further include a pulse generating circuit (not shown) which is configured to generate an electrical pulse so that the ultrasound transduction circuit 104 is triggered to generate and transmit ultrasound waves.

The tissue penetrating portion 101 is of an elongated shape. The tissue penetrating end 102 is of a bevel shape. In one exemplary embodiment, tissue penetrating portion 101 may be a needle or a catheter. In another exemplary embodiment, the tissue penetrating portion 101 may be a tube.

The ultrasonic scanner 103 is of an elongated shape.

The ultrasonic scanner 103 may further include an amplifying circuit (not shown) being configured to amplify the returned sound waves. The ultrasonic seamier 103 further includes a circuit board 105. For example, the circuit board 105 may be a flexible circuit board. The circuit board 105 may be of an elongated shape. The ultrasound transduction circuit 104, the pulse generating circuit, and the amplifying circuit may be coupled to the circuit board 105 such that the ultrasound transduction circuit 104, the pulse generating circuit, and the amplifying circuit are electrically connected with each other via the circuit board 105.

The pulse generating circuit and the amplifying circuit may be assembled together in an application-specific integrated circuit (ASIC) 106, and the ASIC 106 may be coupled to the circuit board 105 such that the ASIC 106 and the ultrasound transduction circuit 104 are electrically connected. The ASIC may only have the function for generating pulse and amplifying echo received. Therefore the cost of the ASIC may be dramatically reduced which may make the device 100 more competitive in disposable usage.

The circuit board 105 may coupled along the tissue penetrating portion 101.

The ultrasound transduction circuit 104 may be coupled to a subject facing end of the ultrasonic scanner 103. In a further embodiment, the subject facing end of the ultrasonic scanner 103 is coupled to the tissue penetrating end 102 of the tissue penetrating portion 101.

The ultrasound transduction circuit 104 may be coupled to a subject facing end of the circuit board 103. The subject facing end of the circuit board 105 may be coupled to the tissue penetrating end 102 of the tissue penetrating portion 101.

Any of the ultrasound transduction circuit 104, the pulse generating circuit, and the amplifying circuit may be coupled to the circuit board 105 via wire-bond or with flip-chip. For example, the ASIC 106 may be coupled to the circuit board via wire-bond or with flip-chip.

The ultrasound transduction circuit 104 may include an array of one or more ultrasound transducers. For example, the ultrasound transduction circuit 104 may includes a linear array of one or more ultrasound transducers.

The tissue penetrating portion 101 may be hollow, and the ultrasonic scanner 103 may be fixed inside and along the tissue penetrating portion 101. Further, the ultrasonic scanner 103 may be fixed inside and along the tissue penetrating portion 101 such that there is at least one passway through the tissue penetrating portion 101.

For example, the ultrasonic scanner 103 may be fixed inside and along the tissue penetrating portion 101 using glue.

The device 100 may be configured to be coupled to a injector (not shown).

The ultrasonic scanner 103 may be configured to receive a signal to initiate the pulse generating circuit to generate the electrical pulse.

The ultrasonic scanner 103 may be configured to output the returned sound waves.

FIG. 1 (b) illustrates the cross section view of the subject facing end of the device 100.

As can be seen, the ultrasound transduction circuit 104 is located at a subject facing end of the ultrasonic scanner 103 and also a subject facing end of the circuit board 105. For example, the pulse generating circuit and the amplifying circuit may be assembled together in a application-specific integrated circuit (ASIC) 106, and the ASIC 106 may be coupled to the circuit board 105 such that the ASIC 106 and the ultrasound transduction circuit 104 are electrically connected.

FIG. 1 (c) illustrates the working mechanism of the ultrasound transduction circuit 104. For example, the ultrasound transduction circuit may contain a linear array of miniaturized ultrasound transducers.

The linear transducer imaging array may be capable of generating phase shifted pulse on each individual element to form focus ultrasound beam 130. Therefore the echo from the objects at the focal zone 110 along the focus beam may be amplified and processed afterward to generate an ultrasonic image. By repeating the same process with different focusing angle, a field of view of approximately +/−45 degree from the normal vector of the linear array surface may be constructed. The image acquired in the process may be displayed on a monitor to show the practitioners of the tissues or organ structure in front of the tissue penetrating end, e.g. a needle tip. The device may be applied, for example, in the application in helping the procedure for epidural anesthesia and for organ tissue sampling.

FIG. 2 (a) illustrates a photo of a device 200 for penetration a tissue of a subject according to an exemplary embodiment. In this exemplary embodiment, the tissue penetrating portion is a rod, and the ultrasonic scanner may be coupled along the rod. The ultrasound transduction circuit is coupled to a subject facing end of the ultrasonic scanner and the subject facing end of the ultrasonic scanner is coupled to the tissue penetrating end. Further, the rod and the ultrasonic scanner are configured to be covered with a sheath catheter upon penetrating the tissue of the subject. The space between the sheath catheter and the tissue penetrating portion may be used for drug delivery or for collection of organ tissue samples, for example.

FIG. 2 (b) illustrates a photo of the device 200 wherein the sheath catheter is dissembled from the rod and the ultrasound transduction unit.

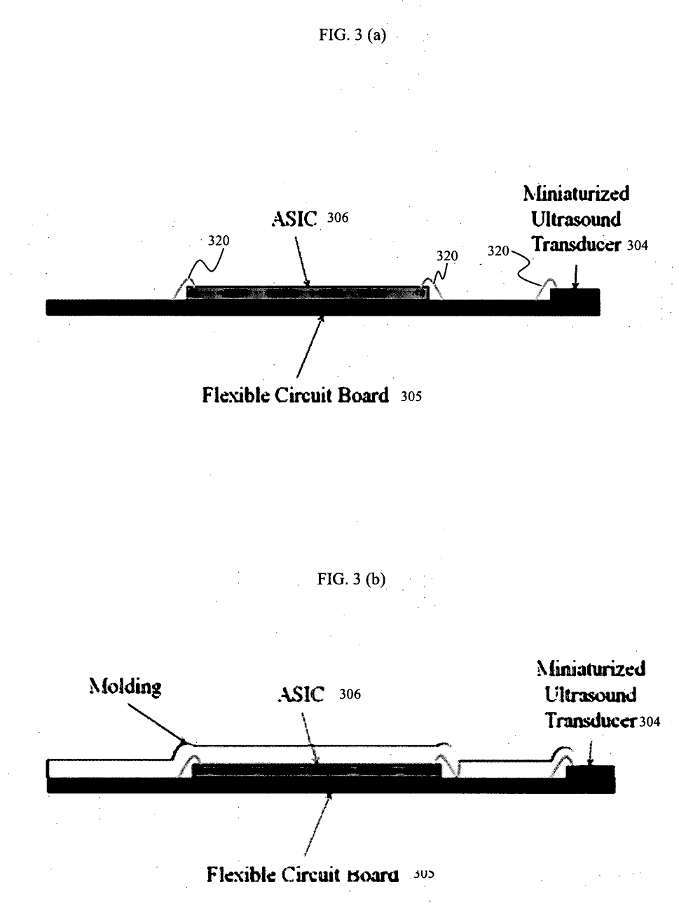

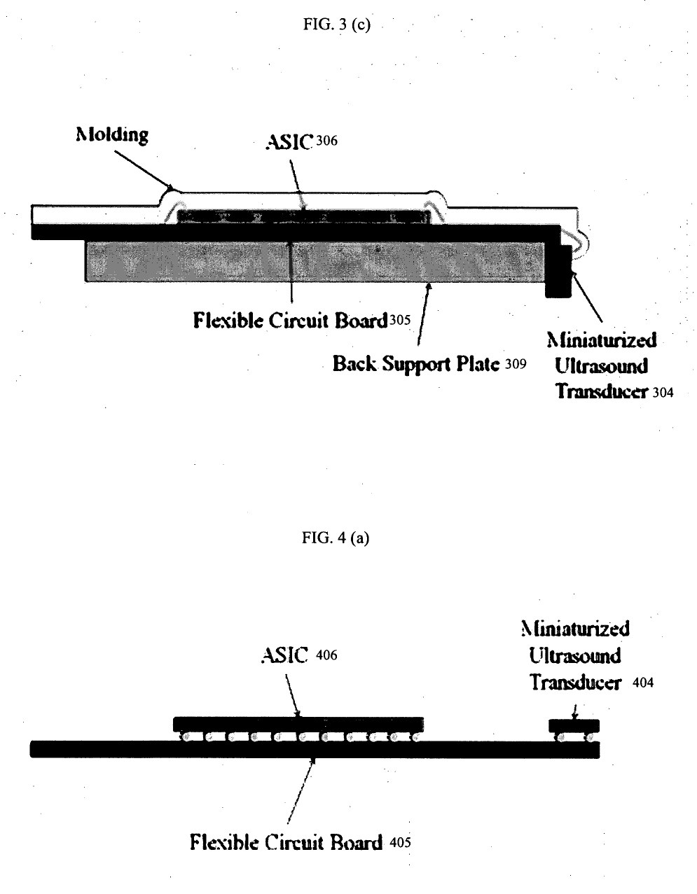

FIGS. 3 (a) to (c) illustrate the assembly process of the ultrasonic scanner according to one exemplary embodiment. The ultrasonic scanner may include a flexible circuit board 305, an ultrasound transducer (ultrasound transduction circuit) 304, an ASIC 306 and a tissue penetrating portion 301 which may for example be a needle or catheter. The pulse generating circuit (not shown) and an amplifying circuit (not shown) may be assembled together in the ASIC 306, and the ultrasound transducer 304 and the ASIC 306 are electrically connected.

FIG. 3 (a) illustrates that the ASIC 306 and miniaturized ultrasound transducer 304 are assembled to the flexible circuit board 305 with wire-bonding (see the wires 320). The miniaturized ultrasound transducer 304 may be in a form of a chip, for example.

FIG. 3 (b) illustrates the bond wire 320, the ASIC 306 and part of the miniaturized ultrasound transducer 304 are molded for protection. The molding material may be any material that can be applied in shape changing form and solidified after molding process. Examples of materials are PDMS, photopolymer or polycarbonate molding rasin. Alternatively, a CVD deposition with paralyne may be applied. The purpose of the molding process is to cover the ASIC, bond-wire 320 and part of the ultrasound transducers 304 for protection.

FIG. 3 (c) illustrates that the flexible circuit board 305 is bended at the portion of the ultrasound transducer chip 304. Further, a back support plate 309 may be glued at the back of the circuit board 305 to increase the rigidity of the board 305.

Thereafter, the circuit board 305 along with ASIC 306 and ultrasound transducer 304 may be inserted into a tissue penetration portion such as a needle or catheter and glued on the inside wall of the tissue penetration portion. The final device may be the one as shown in FIG. 1 (a), for example.

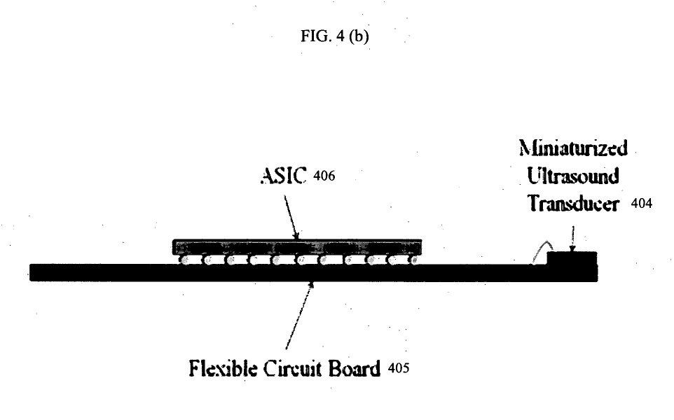

FIGS. 4 (a) to (b) illustrate the electrical interconnection between the circuit board 405 and the ASIC 406 and between the circuit board 405 and the ultrasound transducer 404 may be achieved by flip-chip instead of wire-bond or a mix and match of both type of wire-bond and flip-chip.

FIG. 4 (a) illustrates that the electrical interconnection between the circuit board 405 and the ASIC is achieved by flip-chip, and the electrical interconnection between the circuit board 405 and the miniaturized ultrasound transducer 404 is also achieved by flip-chip.

FIG. 4 (b) illustrates mixed types of electrical interconnection between the circuit board 405 and the ASIC 406 and between the circuit board 404 and the ASIC 406 may be applied.

In FIG. 4 (b), the electrical interconnection between the circuit board 405 and the ASIC 406 is achieved by flip-chip, and the electrical interconnection between the circuit board 405 and the miniaturized ultrasound transducer 404 is also achieved by wire-bond.

In summary, various embodiments provide a device with ultrasound imaging capability is disclosed to facilitate the anesthesia or drug delivery procedure and to facilitate organ tissue samplings. The hassle during the procedure may be greatly reduced since the image may be provided through the device. The space (passway) between the ultrasonic scanner and the tissue penetrating portion provides the passage for drug delivery or for collection of organ tissue samples. The tissue penetrating portion may be a needle or catheter with bevel tips (tissue penetrating end). The needle or the catheter may be a hollow elongated tube or of solid filled rod. The ultrasonic scanner may include, at the subject facing end of the ultrasonic scanner, an ultrasound transduction circuit which contains a linear array of plural ultrasound transducers as an imaging device. The ultrasonic scanner may be inserted into the hollow needle or catheter, and the linear array may be fixed inside the needle or catheter at the subject facing end of the needle or catheter. The linear array inside the needle or catheter may be made of miniaturized ultrasound transducers. An ASIC in which a pulse generating circuit and an amplifying circuit may be assembled may be placed right beside the linear array for driving pulse and amplifying the receiving echo.

In various embodiments, the miniaturized ultrasound transducers may be combined along with a tissue penetrating portion such as a needle. The ultrasound transducer array formed by the miniaturized ultrasound transducers may provide sufficient field of view within the space between disks in the spine by operating at higher frequency with better resolution, for example.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A device for penetrating a tissue of a subject, comprising:
a tissue penetrating portion having a tissue penetrating end;
an ultrasonic scanner coupled to the tissue penetrating portion;
wherein the ultrasonic scanner comprises
an ultrasound transduction circuit being configured to generate and transmit ultrasound waves to the subject and to receive returned sound waves echoed from the subject; and
a pulse generating circuit being configured to generate an electrical pulse so that the ultrasound transduction circuit is triggered to generate and transmit ultrasound waves,
wherein the ultrasonic scanner further comprises a circuit board, wherein the circuit board is coupled along the tissue penetrating portion.

2. The device according to claim 1, wherein the tissue penetrating portion is of an elongated shape or a bevel shape.

3. (canceled)

4. The device according to claim 1, wherein the tissue penetrating portion is a needle, catheter or a tube.

5. (canceled)

6. The device as claimed in claim 1, wherein the tissue penetrating portion is a rod.

7. (canceled)

8. The device as claimed in claim 1, wherein the ultrasonic scanner further comprises an amplifying circuit being configured to amplify the returned sound waves.

9. (canceled)

10. (canceled)

11. The device as claimed in claim 1, wherein the ultrasound transduction circuit, the pulse generating circuit, and the amplifying circuit are coupled with the circuit board such that the ultrasound transduction circuit, the pulse generating circuit, and the amplifying circuit are electrically connected with each other via the circuit board.

12. The device as claimed in claim 1, wherein the circuit board is a flexible circuit board.

13. The device as claimed in claim 11, wherein the pulse generating circuit and the amplifying circuit are assembled together in an application-specific integrated circuit (ASIC), and the ASIC is coupled to the circuit board such that the ASIC and the ultrasound transduction circuit are electrically connected.

14. (canceled)

15. The device as claimed in claim 1, wherein the ultrasound transduction circuit is coupled to a subject facing end of the ultrasonic scanner.

16. The device as claimed in claim 15, wherein the subject facing end of the ultrasonic scanner is coupled to the tissue penetrating end of the tissue penetrating portion.

17. The device as claimed in claim 1, wherein the ultrasound transduction circuit is coupled to a subject facing end of the circuit board.

18. The device as claimed in claim 17, wherein the subject facing end of the circuit board is coupled to the tissue penetrating end of the tissue penetrating portion.

19-22. (canceled)

23. The device as claimed claim 1, wherein the tissue penetrating portion is hollow, and the ultrasonic scanner is fixed inside and along the tissue penetrating portion.

24. The device as claimed in claim 23, wherein the ultrasonic scanner is fixed inside and along the tissue penetrating portion such that there is at least one passway through the tissue penetrating portion.

25. (canceled)

26. The device as claimed in claim 6, wherein the ultrasonic scanner is coupled along the rod.

27. The device as claimed in claim 26, wherein the ultrasound transduction circuit is coupled to a subject facing end of the ultrasonic scanner and the subject facing end of the ultrasonic scanner is coupled to the tissue penetrating end.

28. The device as claimed in claim 27, wherein the rod and the ultrasonic scanner are configured to be covered with a sheath catheter upon penetrating the tissue of the subject.

29. The device as claimed in claim 1, wherein the device is configured to be coupled to a injector.

30. The device as claimed in claim 1, wherein the ultrasonic scanner is configured to receive a signal to initiate the pulse generating circuit to generate the electrical pulse.

31. The device as claimed in claim 1, wherein the ultrasonic scanner is configured to output the returned sound waves.
